(19) United States
(12) Patent Application Publication
(10) Pub. No.: US 2017/0112670 A1
(43) Pub. Date: Apr. 27, 2017

(54) Patient Eye Protection Device For Dermatology, X-Ray, and General Anesthesia Procedures

(71) Applicant: Joe O'Brien, North Oaks, MN (US)

(72) Inventor: Joe O'Brien, North Oaks, MN (US)

(21) Appl. No.: 14/750,431

(22) Filed: Jun. 25, 2015

Related U.S. Application Data
(60) Provisional application No. 62/019,650, filed on Jul. 1, 2014.

(51) Int. Cl. A61F 9/04; B65D 73/00

(52) U.S. Cl. CPC A61F 9/045; B65D 73/0028

(57) ABSTRACT

The present invention relates to an eye protection cover; and in particular, to an eye protection cover that protects the eye during medical or health care treatment procedures. The cover is comprised of a heat resistant material, conforms in three dimensions to the shape of the eye, and includes a handle for ease of insertion and removal.

### RELATED APPLICATIONS

The present application claims priority to and incorporates by reference U.S. Provisional Patent Application No. 62/019,650 filed on Jul. 1, 2014. The present application incorporates by reference U.S. patent application Ser. No. 13/786,897 filed on Mar. 6, 2013.

### BACKGROUND OF THE INVENTION

Field

The present invention relates to an eye protection cover; and in particular, to an eye protection cover that protects the eye during medical or health care treatment procedures. The cover is comprised of a heat resistant material, conforms in three dimensions to the shape of the eye, and includes a handle for ease of application or insertion and removal.

Background

Dermatology & Aesthetics

Common dermatological and aesthetic procedures such as laser treatments, intense pulsed light, LED therapy, microdermabrasion, and others present potential hazards to the eyes of the patient.

The optical energy from light based devices such as laser, intense pulse light (IPL), and LED therapy present an eye hazard related to the energy inherent in these treatment modalities. Optical energy from these devices is absorbed by the cornea and/or retina and may damage the vision of the patient.

Other dermatological therapies such as microdermabrasion and chemical treatments can cause mechanical or chemical damage to the eyes of the patient.

Protection of the patient's eyes during dermatological and aesthetic procedures can be challenging, particularly when the procedures are performed around the eyes and face. Traditional protective eyewear and masks often sit too far away from the eye, leaving a risk of damage to the eyes. Many procedures are performed close to the eyes. Traditional eyewear is often too big and bulky, and interferes with the treatment.

A small disposable eye patch is disclosed in U.S. Pat. No. 6,320,094 9 (Arnold et al). In this patent, Arnold et al. discloses a method of layering sheets of a foam material with an adhesive layer exposed at the outer edge. For certain applications, such as laser, a foil layer is included as a middle layer to provide sufficient protection. This product is commercially available in the marketplace.

The Arnold et al. device, however, suffers from a number of shortcomings, and in particular relating to ease of use and effectiveness. The outer edges of the device are comprised of a foam material known to have a low melting point. If a laser were to contact this material, it is likely that the energy from the laser would melt the material, and likely burn the patients. Furthermore, the eye-cover is two dimensional and therefore does not properly confirm to the contour of the eyes, which causes coverage issues and issues relating to comfort. Furthermore, the device is often applied by a physician or other health care profession who would be wearing gloves. The adhesive on this device sticks to the gloves (or to a person's hands even if gloves were not worn) making the device difficult to apply and difficult to reposition on the patient's eyes.

FIG. 1 shows a commercial embodiment of the Arnold et al. device. The device is affixed to a backer layer. A protruding grip is provided that can be used to peel the device from backer. The adhesive which secures the device to the backer is used to secure the device over a patient's eyes. As mentioned, the device is planer in nature and fails to adequately follow the three dimensional contours of the eye. Also, the device is difficult to handle since it requires touching the adhesive, which sticks especially to gloves. Further, the device is comprised of foam which has a very low melting point.

X-Ray Procedures

It is known that exposure to ionizing radiation can lead to cataracts. The exact mechanisms of radiation related cataracts are not fully understood. These cataracts may occur far removed from the treatment. X-ray radiation, especially repeated exposure, can lead to other unwanted complications. Therefore, an improved and simple device for protecting the eyes of a patient from harm such as that produced by X-rays is desirable.

General Anesthesia

Eye injuries to patients who undergo general anesthesia are somewhat common. While a number of more serious eye injuries can result from general anesthesia, the most common injury is corneal abrasion. Although less serious than corneal abrasion, post surgical eye irritation is extremely common.

The cornea is the superficial clear layer of cells on the eyeball. A corneal abrasion is a scratch or tear in the cornea. Corneal abrasions can cause mild to severe discomfort, irritation, and pain. Additionally, corneal abrasion may also effect normal vision. While corneal abrasions often heal without long-term effects on vision, severe abrasions may leave a scar on the cornea which may be noticed as a dark or blurred spot in normal vision.

During general anesthesia, the eyelids of some patients do not fully close or reflexively blink to keep the cornea moist. This occurs in approximately 6 out of 10 people. The dry cornea may stick to the inside of the eyelid, causing an abrasion when the eye opens post surgery. Corneal abrasion may also occur if something brushes against the open eye during surgery. This could be a surgical drape or the patient's own fingers when confused post surgically, or any other object that passes near to the eye.

Medical professionals take care to avoid corneal abrasions during surgery. Gel is often applied to the eyes, and the eyes are typically taped closed using gauze and tape. Some adhesive eye patch devices exist in the marketplace to prevent such injuries. These measures and devices, however, are not always sufficient, because corneal abrasions and other eye injuries associated with surgery and medical procedures are still common. Furthermore, the application of gauze and tape to protect the eye makes it impossible for the anesthesiologist to easily view the patient's pupils, which is an important part of anesthesia management.

Despite these best efforts injuries and irritation do take place. Simply using gel and gauze pads still fails to eliminate post surgical eye redness, irritation and pain in up to 50% of surgical cases. These symptoms, typically resolve in a matter of hours, but can be range from mildly to very irritating and painful.

Studies have shown that serious corneal abrasions requiring treatment occur in as many as 1 in 1,750 surgical cases. In certain procedures, including brain and spinal surgery, this number has been described as 1 in 580 procedures. Under microscopic investigation, microscopic corneal abrasions occur in as many as 1 in 25 surgical procedures have been discovered.

Accordingly, there is a need for an improved eye protection device that eliminates or substantially eliminates the drawbacks of the prior art.

### BRIEF DESCRIPTION OF THE FIGURES

FIG. 1 shows a prior art eye cover.

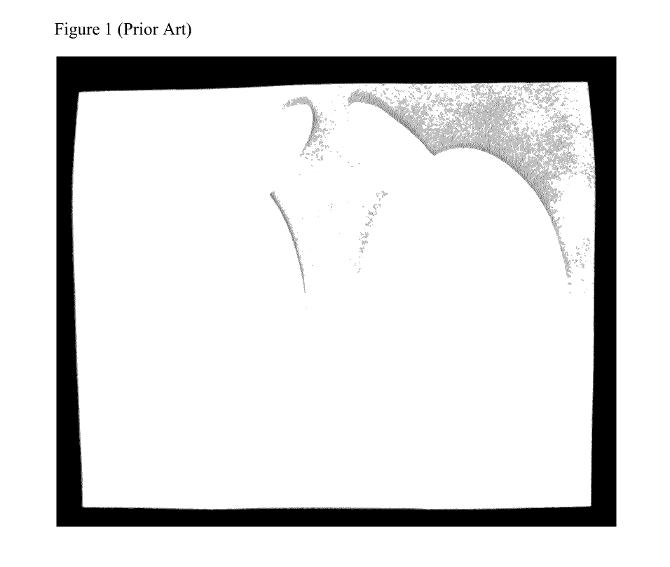

FIGS. 2a and 2b show an eye cover of the present invention.

FIG. 3 shows a top view of the eye cover of the present invention.

FIG. 4 shows a bottom view of the eye cover of the present invention.

FIG. 5 shows a side view of the eye cover of the present invention.

### DETAILED DESCRIPTION OF THE INVENTION

The purpose of the present invention, in its various forms, is to protect the eyes of a patient undergoing dermatological procedures, x-ray procedures, general anesthesia, as well as other treatments such as those common to health spas such as tanning, facials, and other similar and related fields of use.

The invention consists of an injection molded silicone eye cup, or cover. The eye cover is three dimensionally shaped to fit within the eye socket of the patient.

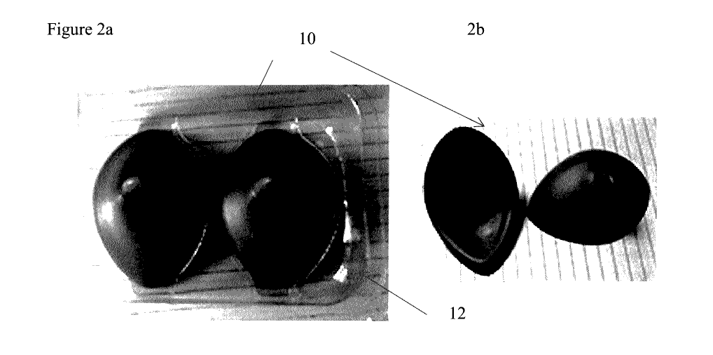

FIG. 2a shows a top view of the eye cover 10. The cover is initially provided releaseably secured to a casing 12, such as the plastic casing shown in FIG. 2a. The eye cover can be removed from the casing just prior to application. Alternatively, they eye cover 10 can be packaged in a heat sealed foil pouch, which eliminates the need for a releasably secured casing. The outer portion of the inside edge of the eye cover 10 is coated with an adhesive, which will also hold the eye cover 10 in place when applied to a patient's eyes. FIG. 2b shows the eye cover in two orientations. First, the eye cover 10 is shown from the bottom in the view on the left of FIG. 2b. The right side of FIG. 2b shows the eye cover 10 from the top.

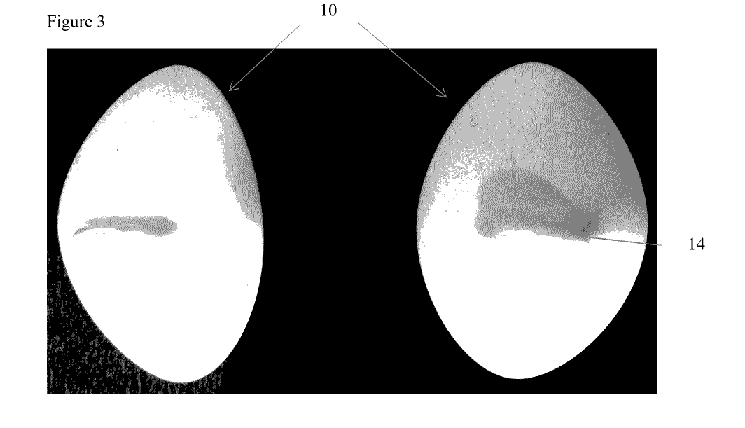

FIG. 3 shows a top view of two eye covers 10. As can be seen, the eye cover 10 is generally oval, or tear drop shaped, with tapered ends, which conform to the shape of the eye. The eye cover 10 is also curved both along a transverse and longitudinal axis thereby forming a concave shape to further conform to the shape of the eye socket.

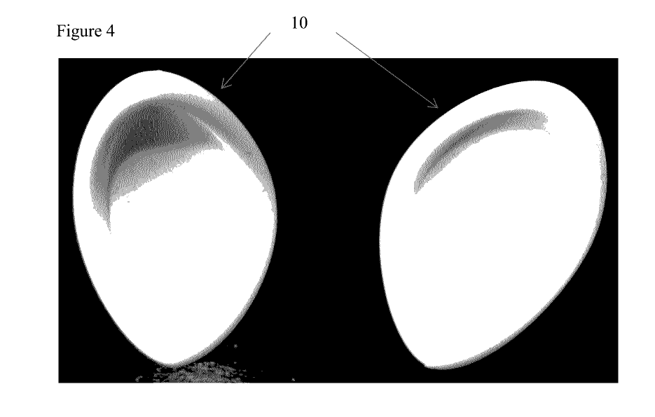

FIG. 4 shows the eye cover 10 from the bottom. The outer curved edge is adapted for application of an adhesive that can secure the eye cover 10 to packaging, as well as to a patient.

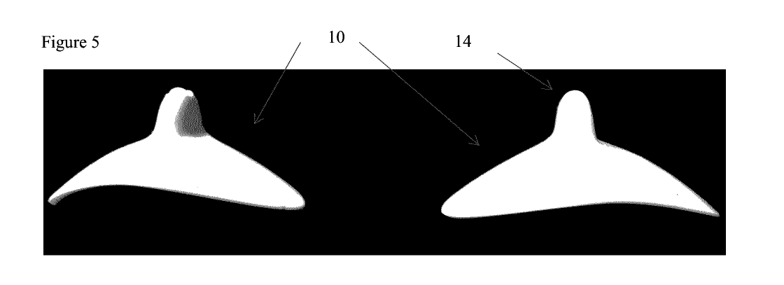

FIG. 5 shows the eye cover 10 from the side, wherein the concave shape of the eye cover 10 can be clearly seen. The eye cover 10 also includes a protruding centrally located handle 14 that can be used to easily insert and remove the eye cover 10, especially when gloves are worn since the handle does not have any adhesive on it.

At the edge of the underside of the eye cover 10, which contacts the skin around the eyes, a 2-part silicone gel adhesive is applied. The adhesive is designed to hold the eye cover 10 in place on the eyes of the patient, and allow the device to be removed and re-applied if necessary, which is a problem with currently marketed devices.

The eye cover 10 is further shaped to follow the curvature of the eyelid. This overcomes a problem with prior art devices, which are flat. It is sometimes difficult for the physician to get these sheet type eye protectors to lie flat over the eye.

Because of the high melting point of the silicone material, the eye cover 10 of the present invention is well suited for protection against optical devices, such as lasers. These devices generate heat when they contact material. Currently used materials may melt at low temperatures, causing burn injuries around the patient's eyes, and require other high temperature materials such as foils to be incorporated, which increases the complexity and cost of prior art devices as compared to the present invention. The high melting point of silicone gives the present invention excellent resistance against lasers and other light energy devices. In one embodiment of the invention, the eye cover 10 is also comprised of carbon black pigment which gives the eye cover 10 a very high optical density.

Further, a small handle or holder 14 is configured on the outward facing side of the device. This handle 14 allows the physician to easily apply, remove and re-apply the eye cover 10 without contacting the adhesive. This overcomes a problem with prior art devices, which are difficult to apply without the adhesive sticking to the physician's gloves.

Still further, the concave cup like shape of the present invention provides for additional flexibility to adapt to variations in the size and shape of different patient's eyes. Thus, allowing the eye cover 10 to be more easily used in a wider array of circumstance.

In an additional embodiment of the device, a radiopaque material, such as barium or bismuth could be blended with the silicone prior to molding. The radiopaque material would provide protection from x-rays.

In a still further embodiment of the device a small sponge-like or otherwise absorbent material could be placed inside of the device, so as to be positioned in front of the eye. This material could be saturated with a tear replacement solution prior to application. During the procedure, the micro-climate in front of the eye would be maintained at high humidity, to prevent the eye from drying out thereby preventing associated injury to the eye.

Additionally, when the eye cover 10 is used in combination with anesthesia the eye cover 10 would be comprised of a transparent silicon material that would allow for a direct view of the patient's eye/pupil through the eyelids.

Further embodiments of the present invention include connecting two eye covers 10 with a bridge piece to provide a joined eye cover 10 that can cover both eyes. The joined eye cover 10 can also comprise a strap or bands that go around all or a portion of the patient's head for more secure attachment. The eye cover 10 can be prepackaged in plastic as shown herein, or in a foil package, or have a removable backer cover attached to the adhesive, or in some combination thereof. Additionally, the handle position and configuration can vary from what is shown in the Figures. The handle can be rotated 90° from the position shown in the figures. The handle can be asymmetrically located away from the center of the eye cover. Also, the eye cover can include more than one handle.

The present invention substantially overcomes the problems of the prior art by providing an eye cover 10 that is flexible, anatomically correctly shaped, is easy to apply and remove, and that can effectively keep the eyes moist during anesthesia without causing or allowing some occurrence of corneal abrasion. It is anticipated that the eye cover 10 of the present invention will more effectively protect the eyes from optical, x-ray, thermal, and mechanical injury. It is anticipated that the invention will prevent most or all eye injuries related to the above mentioned risks.

These and other advantages will be apparent to those of ordinary skill in the art.

While the various embodiments of the invention have been described in reference to the Figures, the invention is not so limited. Also, the method and apparatus of the present invention is not necessarily limited to dermatology or medical procedures, but can be applied to any field where covering the eyes is warranted.

Unless otherwise defined, all technical and scientific terms used herein have the same meaning as commonly understood by one of ordinary skill in the art to which this invention belongs. Although methods and materials similar to or equivalent to those described herein can be used in the practice or testing of the present invention, suitable methods, and materials are described below. All publications, patent applications, patents, and other references mentioned herein are incorporated by reference in their entirety to the extent allowed by applicable law and regulations. In case of conflict, the present specification, including definitions, will control.

The present invention may be embodied in other specific forms without departing from the spirit or essential attributes thereof, and it is therefore desired that the present embodiment be considered in all respects as illustrative and not restrictive, reference being made to the appended claims rather than to the foregoing description to indicate the scope of the invention. Those of ordinary skill in the art that have the disclosure before them will be able to make modifications and variations therein without departing from the scope of the invention.

1. An eye cover comprising:
a casing having a three dimensional contour conforming generally to the shape of an eye.

2. The eye cover of claim 1 further comprising an adhesive layer for releasable securement to an eye.

3. The eye cover of claim 1 further comprising a handle.

4. The eye cover of claim 1 further comprising an adhesive layer for releasable securement to an eye, and a non-adhesive handle.

5. The eye cover of claim 1 wherein the eye cover is secured to a casing with releasable adhesive.

6. The eye cover of claim 1 wherein the eye cover is packaged in a pouch.

7. The eye cover of claim 1 wherein the eye cover is concave in shape.

8. The eye cover of claim 1 wherein the eye cover has an outer raised curved edge.

9. The eye cover of claim 9 wherein the edge is coated with a releasable adhesive.

10. The eye cover of claim 3 where the handle protruded outward from the center of the eye cover.

11. The eye cover of claim 1 wherein the eye cover is comprised of silicone.

12. The eye cover of claim 11 wherein the silicone has a high melting point.

13. The eye cover of claim 1 wherein the eye cover has a high optical density.

14. The eye cover of claim 1 wherein the eye cover is transparent.

15. The eye cover of claim 1 wherein the eye cover is radiopaque.

16. The eye cover of claim 1 further comprising a liquid composition to maintain a high humidity environment inside the eye cover.

17. The eye cover of claim 1 further comprising a second eye cover joined to the first eye cover to cover both eyes of a patient.

18. The eye cover of claim 17 further comprising a strap to secure the joined eye covers to the patient's head.
